(19) United States
(12) Patent Application Publication
(10) Pub. No.: US 2018/0104250 A1
(43) Pub. Date: Apr. 19, 2018

(54) Formulations for treatment of migraines

(71) Applicant: Knowles Enterprises Inc., Miramar, FL (US)

(72) Inventor: Andrew G. Knowles, Miramar, FL (US)

(21) Appl. No.: 15/782,284

(22) Filed: Oct. 12, 2017

Related U.S. Application Data
(60) Provisional application No. 62/408,351, filed on Oct. 14, 2016.

(51) Int. Cl. A61K 31/525; A61K 33/08; A61K 36/28; A61K 47/18

(52) U.S. Cl. CPC A61K 31/525; A61K 47/18; A61K 36/28; A61K 33/08

(57) ABSTRACT

Formulations and compositions for the treatment of migraine headaches, and in general, pain, include riboflavin, magnesium, feverfew and an amino acid, such as 5-hydroxytryptophan (5-HTP).

### CROSS-REFERENCE

In accordance with 37 C.F.R. § 1.76, a claim of priority is included in an Application Data Sheet filed concurrently herewith. Accordingly, the present invention claims priority to U.S. Provisional Patent Application No. 62/408,351, entitled “FORMULATIONS FOR TREATMENT OF MIGRAINES”, filed on Oct. 14, 2016. The contents of the above referenced application is herein incorporated by reference in its entirety.

### FIELD OF THE INVENTION

The present invention relates to pharmaceutical treatments for pain, and in particular to severe headache; to formulations for treatment of migraine headaches, associated symptoms and generally for pain relief; and more particularly to a formulation which includes a combination of riboflavin, magnesium, feverfew, and 5-hydroxytryptophan.

### BACKGROUND

There are two major types of migraines. The common migraine affects 80-85% of migraine sufferers and classical migraine with aura affects 15% of migraine sufferers. The common migraine is typically associated with various psychological (e.g., irritability, depression, fatigue, drowsiness, and restlessness), neurological (e.g., photophobia, and phonophobia), and gastrointestinal symptoms. The headache starts with mild pain, which increases in intensity over a short period of time. In some cases, early management of the headache can reduce the duration and severity of the pain. Headaches in classical migraines are typically characterized by a neurological deficit known as an aura. Exemplary deficits include visual scotoma or visual designs, hemiplegia, migrating paraesthesia, dysarthria, dysphasia, and deja-vus. The headache is usually accompanied by light or sound sensitivity, photophobia or phonophobia, irritability and impaired concentration. Treatment of the classical migraine at the time of the aura may alleviate the severity and duration of the headache.

Currently available drugs to alleviate the pain associated with migraines have modest or limited efficacy and are associated with various debilitating side effects. Thus, better therapies are needed for the management of migraines.

### SUMMARY

Embodiments are directed to prevention and treatment of migraine headaches, associated symptoms thereof, and in general, other forms of pain. In particular, a composition comprises riboflavin, magnesium, feverfew, 5-hydroxytryptophan (5-HTP) or combinations thereof.

In one embodiment, the invention includes a therapeutic composition comprising riboflavin, magnesium, feverfew, an amino acid, an amino acid precursor, or combinations thereof. The therapeutic composition may comprise about 10% to about 70% weight/weight (w/w) of riboflavin, about 10% to about 60% w/w of magnesium, about 5% to about 30% w/w of feverfew, about 5% to about 30% w/w of 5-hydroxytryptophan (5-HTP) or combinations thereof. The therapeutic composition may comprise a unit dose of about 100-500 mg of riboflavin, about 100-400 mg magnesium oxide and/or magnesium salt of an organic salt, about 20-200 mg of feverfew, about 20-200 mg of 5-hydroxytryptophan (5-HTP) or combinations thereof.

In another illustrative embodiment, the invention includes a therapeutic composition for treatment or prevention of migraines consisting essentially of riboflavin, magnesium, feverfew, and 5-hydroxytryptophan (5-HTP). The therapeutic composition may consists of about 10% to about 70% weight/weight (w/w) of riboflavin, about 10% to about 60% w/w of magnesium, about 5% to about 30% w/w of feverfew and about 5% to about 30% w/w of 5-hydroxytryptophan (5-HTP). The therapeutic composition may comprise a unit dose consisting of about 100-500 mg of riboflavin, about 100-400 mg magnesium oxide and/or magnesium salt of an organic salt, about 20-200 mg of feverfew and about 20-200 mg of 5-hydroxytryptophan (5-HTP).

In another illustrative embodiment, the invention includes a composition for the prevention and treatment of migraine headaches and associated symptoms thereof comprising: an effective amount of riboflavin; an effective amount of magnesium provided as magnesium oxide, magnesium salt of an organic salt, or combinations thereof; an effective amount of feverfew or feverfew extract; an effective amount of 5-hydroxytryptophan (5-HTP); and an effective amount of at least one pharmaceutical acceptable carrier.

In another illustrative embodiment, the invention includes a method of treating or preventing a migraine comprising, administering a therapeutic composition to a subject in need thereof, the therapeutic composition comprising about 10% to about 70% weight/weight (w/w) of riboflavin, about 10% to about 60% w/w of magnesium, about 5% to about 30% w/w of feverfew, about 5% to about 30% w/w of 5-hydroxytryptophan (5-HTP) or combinations thereof.

### DETAILED DESCRIPTION

As a new over-the-counter (OTC) medication, the present invention provides substantial benefits to subjects suffering from migraine headaches, not least of which may be the cost savings associated with a decreased reliance on expensive prescription medications and the reduction in the economic burden of migraine in the United States. There are also substantial advantages for those individuals who choose to rely on OTC medications for relief of migraine headache, namely the availability of another treatment option that may be more effective than currently available OTC products, or which associated with fewer side effects than currently available OTC products.

Accordingly, embodiments of the invention are directed to formulations and compositions thereof, for the prevention and treatment of migraine headaches and associated symptoms thereof, headaches, tension headaches, neuropathic pain and pain in general. The formulations embodied herein, comprise riboflavin, magnesium, feverfew, 5-hydroxytryptophan (5-HTP) or combinations thereof.

### Definitions

Unless defined otherwise, all technical and scientific terms used herein have the same meaning as commonly understood by one of ordinary skill in the art to which the invention pertains. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “composition” is used herein to include formulations which are intended for co-administration, either sequentially or simultaneously. It is generally more convenient, however, for the composition to be in a single admixture formulation. The components of the composition can be mixed prior to ingestion, e.g., within sixty seconds of ingesting, or administered separately in a time that typically does not exceed five minutes.

An “effective amount” as used herein, means an amount which provides a therapeutic or prophylactic benefit.

The term “extract”, as used herein includes any preparation obtained from plants, fruits or vegetables using an extraction method.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein the term “topical formulation” refers to a formulation that may be applied to skin or a mucosa surface. Topical formulations may, for example, be used to confer therapeutic benefit to a patient. Topical formulations can be used for both surface (non-transdermal) as well as transdermal (penetrating the skin or mucosal surface) administration of substances.

The term “topical administration” is used in its conventional sense to mean delivery of a substance, such as a therapeutically active agent, to the skin, mucosal surface, or a localized region of the body. Topical administration of a local anesthetic drug, for example, may often be advantageously applied during or prior to, for example, a painful medical or cosmetic procedure or to numb or otherwise treat the skin, or to reduce or eliminate an already existing pain.

As used herein, the term “transdermal” means in the broadest sense through the skin.

The term “transdermal administration” is used to mean administration through the skin or mucosal surface. Transdermal administration is often applied where systemic delivery of an active is desired, or alternatively, it may also be useful for delivering an active to tissues underlying the skin with minimal systemic absorption (i.e. localized delivery).

As used herein, the term “amino acid” may be used to define an organic compound containing amine (—NH2) and carboxyl (—COOH) functional groups, along with a side chain (R-group) specific to each amino acid.

The term “amino acid precursor” may be used to define compounds which give amino acids after some reaction, usually a hydrolysis reaction.

### Migraine and Pain Relieving Formulations

Some migraine headaches are preceded by aura or other symptom, whereby treatment of the symptom may prevent the further development of the migraine headache. The present invention provides surprisingly rapid relief, so that certain patients may avoid full onset of migrainous headaches and their associated symptoms through immediate administration of the present treatment upon onset of this symptom. In addition, many associated symptoms of migraine are found to be relieved, such as nausea, vomiting, photophobia and phonophobia (excessive/extreme sensitivity to light in one case and sound in the other). There is a growing body of knowledge which suggests that headache exists along a spectrum, with no rigid demarcation between “migraine” and “non-migraine,” and that many headaches previously thought to be “not migraine” are in fact “migrainous.” This may explain the fact that the present invention is effective in the treatment not only of headaches experienced by those not having been previously diagnosed with migraine, and whose headaches may not specifically qualify as migraine per the presently accepted diagnostic criteria of the International Headache Society, but also headaches not so diagnosed, including as but one example those associated with excessive alcohol use (e.g. “hangovers”).

The compositions embodied herein are directed to preventing and treating migraine headaches, and in general neuropathic pain including any indication, condition, disorder or disease that involves neuropathic pain, including migraine, neuropathic pain of diabetes, diabetic neuropathy (e.g., diabetic peripheral neuropathy), shingles, burn injuries, ophthalmic injuries, oral nerve injury, reflex sympathetic dystrophy (RSD), post-herpetic neuralgia, arthritis, or neuropathic pain by injury, amputation or overuse, including sciatica and low back pain. The compositions and methods for treating migraine headaches and associated symptoms thereof, including neuropathic pain, presented herein alleviate (e.g., ameliorate, attenuate, reduce or diminish) at least one symptom of migraine pain, including neuropathic pain, such as, for example, allodynia, hyperesthesia, hyperalgesia, spontaneous burning pain, or phantom pain syndrome. Such symptoms may be characteristic of, associated with, or arising out of the pain syndrome. Symptoms from nerve injury or damage, including sensory nerve injury or damage, are pain, including pain that persists after signs of original injury or damage disappear (e.g., neuropathic pain), numbness, tingling, burning and/or loss (e.g., dullness) of feeling. Compositions and methods of the present invention are useful for the alleviation (e.g., amelioration, attenuation, reduction or diminishment) of such symptoms.

Compositions according to the invention comprise riboflavin, magnesium, feverfew, an amino acid, an amino acid precursor or combinations thereof. In certain embodiments, the amino acid is 5-hydroxytryptophan (5-HTP). In certain embodiments, the compositions comprise about 10% to about 70% weight/weight (w/w) of riboflavin, about 10% to about 60% w/w of magnesium, about 5% to about 30% w/w of feverfew or feverfew extract, about 5% to about 30% w/w of 5-hydroxytryptophan (5-HTP) or combinations thereof. In other embodiments, the composition comprises: about 20-800 mg of riboflavin, about 50-600 mg magnesium oxide and/or magnesium salt of an organic salt, about 20-200 mg of feverfew or feverfew extract, about 20-200 mg of 5-hydroxytryptophan (5-HTP) or combinations thereof. In certain embodiments, the composition comprises: about 200 mg of riboflavin, about 180 mg magnesium oxide and/or magnesium salt of an organic salt, about 50 mg of feverfew or feverfew extract, about 50 mg of 5-hydroxytryptophan (5-HTP) or combinations thereof.

In other embodiments, a unit dose comprises: about 50-800 mg of riboflavin, about 50-600 mg magnesium oxide and/or magnesium salt of an organic salt, about 20-200 mg of feverfew or feverfew extract, about 20-200 mg of 5-hydroxytryptophan (5-HTP) or combinations thereof. In other embodiments, a unit dose comprises: about 200 mg of riboflavin, about 180 mg magnesium oxide and/or magnesium salt of an organic salt, about 50 mg of feverfew or feverfew extract, about 50 mg of 5-hydroxytryptophan (5-HTP) or combinations thereof. In certain embodiments, a unit dose consists of about 200 mg of riboflavin, about 180 mg magnesium oxide and/or magnesium salt of an organic salt, about 50 mg of feverfew or feverfew extract and about 50 mg of 5-hydroxytryptophan (5-HTP).

Riboflavin:

In embodiments of the present invention, the migraine treatment formulation comprises riboflavin. Riboflavin is commonly known as vitamin B2 and has been found to improve magnesium transport across the mitochondrial membrane. In embodiments of the present invention, riboflavin is found ranging between 100 milligrams to about 800 milligrams of the formulation. In other embodiments, the riboflavin is found ranging from about 100 mg to about 500 mg. Additional compounds that can be used include riboflavinyl glucoside, riboflavin 5′ phosphate, riboflavin 5′ adenosine diphosphate, riboflavin acid esters, riboflavin butyrate, riboflavin sodium phosphate, riboflavin 5′-phosphate sodium, flavinadeninedinucleotide and flavinmononucleotide.

In some embodiments, a composition provided herein comprises between about 1% and 80% by weight of riboflavin. For example, about 2% to about 80% by weight, about 4% to about 80% by weight, about 5% to about 80% by weight, about 6% to about 80% by weight, about 10% to about 80% by weight, about 20% to about 80% by weight, about 30% to about 80% by weight; about 1% to about 70% by weight, about 1% to about 69% by weight, about 1% to about 68% by weight, about 1% to about 67% by weight, about 1% to about 66% by weight, about 1% to about 65% by weight, about 1% to about 64% by weight, about 1% to about 63% by weight, about 1% to about 62% by weight, about 1% to about 61% by weight, about 1% to about 60% by weight, about 5% to about 70% by weight, about 6% to about 70% by weight, about 7.5% to about 70% by weight, and about 8% to about 70% by weight of a composition provided herein can be riboflavin. In some embodiments, a composition provided herein can be formulated to contain an amount of riboflavin of between about 10% to about 70% by weight (e.g., about 10% to about 69% by weight, about 15% to about 68% by weight, about 16% to about 66% by weight, about 20% to about 65% by weight, about 25% to about 64% by weight, and about 30% to about 60% by weight).

Typically, a composition provided herein comprises between about 20 mg to about 800 mg of riboflavin (e.g., about 25 mg to about 250 mg, about 25 mg to about 200 mg, about 25 mg to about 150 mg; about 50 mg to about 700 mg, about 80 mg to about 700 mg, about 85 mg to about 700 mg, about 90 mg to about 700 mg, about 95 mg to about 700 mg, about 100 mg to about 700 mg, about 125 mg to about 700 mg, about 150 mg to about 700 mg, about 200 mg to about 700 mg; about 50 mg to about 250 mg, about 75 mg to about 250 mg, about 80 mg to about 250 mg, about 90 mg to about 250 mg, about 95 mg to about 250 mg). In some embodiments, a composition provided herein can contain between about 50 mg to about 350 mg of riboflavin (e.g., about 75 mg to about 325 mg, about 80 mg to about 310 mg, about 90 mg to about 300 mg, about 95 mg to about 250 mg).

A composition provided herein can be formulated to comprise an amount of riboflavin such that a daily dose of between about 100 mg and about 1200 mg (e.g., between about 100 and about 1000 mg, between about 100 mg and about 800 mg, between about 100 mg and about 600 mg, between about 100 mg and about 500 mg, between about 100 mg and about 450 mg, between about 200 mg and about 1200 mg, between about 250 mg and about 1200 mg, between about 300 mg and about 1200 mg, between about 350 mg and about 1200 mg, between about 600 mg and about 1200 mg, between about 800 mg and about 1200 mg, between about 200 mg and about 600 mg, between about 250 mg and about 550 mg, between about 300 mg and about 500 mg, between about 350 mg and about 450 mg, and between about 375 mg and about 425 mg) of riboflavin is administered. In some embodiments, a composition provided herein can be formulated to contain an amount of riboflavin such that a daily dose of between about 200 mg and about 600 mg (e.g., between about 250 mg and about 550 mg, between about 300 mg and about 500 mg, between about 350 mg and about 450 mg, and between about 375 mg and about 425 mg) is administered.

Magnesium:

The soluble magnesium compound is provided as a source of magnesium, Mg²⁺ that is rapidly absorb through the lining of the user's stomach. Migraine sufferers have been linked with magnesium deficiencies that affect the onset and duration of migraine symptoms. By providing a supplemental source of magnesium in a fast acting orally administered formulation, the present invention is able to reduce or stop the onset of migraine symptoms within minutes of administering a dosage. The soluble magnesium compound is provided in a manner that reduces gastrointestinal distress commonly associated with the consumption of the magnesium. The soluble magnesium compound accomplishes through a composition that facilitates the absorption rate of magnesium across the stomach lining preventing large amount of magnesium from entering into the small intestine. In the current embodiment of the present invention, the soluble magnesium compound is found ranging from about 50 milligrams to about 600 milligrams of the formulation. In certain embodiments of the present invention, a dosage of the migraine treatment formulation comprises magnesium in a range from about 100 milligrams to about 400 mg dissolved magnesium. It should be noted that the soluble magnesium compound may comprise magnesium containing composition that include but are not limited to, magnesium oxide, magnesium chloride, magnesium citrate, magnesium gluconate, magnesium glycinate, magnesium threonate, magnesium stearate, magnesium salt of an organic salt as well as any magnesium salt derived utilizing an amino acid constituent.

Any biologically useful form of magnesium can be used. For example, magnesium sulfate, magnesium hydroxide, magnesium chloride, magnesium oxide, magnesium gluconate, magnesium malate, magnesium orotate, magnesium glycinate, magnesium citrate, and magnesium lactate can be used. In some cases, the magnesium can be in the form of a magnesium amino acid polysaccharide complex (also known as Magnesium Oligo) as described in U.S. Pat. No. 8,273,393, which is incorporated by reference in its entirety. In some cases, magnesium or a salt or complex containing magnesium can be synthesized or obtained commercially.

In some cases, a composition provided herein comprises between about 5% and 75% by weight of a salt or complex containing magnesium. For example, about 10% to about 75% by weight, about 25% to about 75% by weight, about 32.5% to about 75% by weight, about 35% to about 75% by weight, about 37.5% to about 75% by weight, about 40% to about 75% by weight, about 42.5% to about 75% by weight, about 45% to about 75% by weight, about 50% to about 75% by weight, about 55% to about 75% by weight, about 15% to about 70% by weight; about 15% to about 65% by weight, about 15% to about 60% by weight, about 15% to about 55% by weight, about 15% to about 50% by weight, about 15% to about 47.5% by weight, about 15% to about 45% by weight, about 15% to about 42.5% by weight, about 20% to about 60% by weight, about 25% to about 50% by weight, about 30% to about 50% by weight, about 35% to about 50% by weight, about 40% to about 45% by weight, about 37.5% to about 47.5% by weight, and about 42.5% to about 47.5% by weight of the composition can be a salt or complex containing magnesium. In some embodiments, a composition provided herein can be formulated to contain an amount of a salt or complex containing magnesium of between about 10% to about 60% by weight (e.g., about 25% to about 50% by weight, about 30% to about 50% by weight, about 35% to about 50% by weight, about 40% to about 45% by weight, about 37.5% to about 47.5% by weight, and about 42.5% to about 47.5% by weight). For example, a composition provided herein can contain about 40% by weight of a salt or complex containing magnesium.

Typically, a formulation provided herein comprises between about 25 mg to about 250 mg of magnesium (e.g., about 25 mg to about 200 mg, about 25 mg to about 175 mg, about 25 mg to about 150 mg, about 25 mg to about 120 mg, about 25 mg to about 115 mg, about 25 mg to about 110 mg, about 25 mg to about 100 mg, about 25 mg to about 75 mg, about 25 mg to about 50 mg, about 50 mg to about 250 mg, about 75 mg to about 250 mg, about 80 mg to about 250 mg, about 85 mg to about 250 mg, about 90 mg to about 250 mg, about 95 mg to about 250 mg, about 100 mg to about 250 mg, about 125 mg to about 250 mg, about 150 mg to about 250 mg, about 200 mg to about 250 mg, about 50 mg to about 150 mg, about 75 mg to about 125 mg, about 80 mg to about 120 mg, about 90 mg to about 110 mg, about 95 mg to about 105 mg). In some embodiments, a formulation provided herein can contain between about 50 mg to about 200 mg of magnesium (e.g., about 75 mg to about 180 mg, about 80 mg to about 120 mg, about 90 mg to about 110 mg, about 95 mg to about 105 mg). For example, a composition provided herein can contain about 400 mg of a salt or complex containing magnesium. In some embodiments, the magnesium may be present as part of a salt or complex containing the magnesium. The salt or complex containing the magnesium will have a higher weight as compared to the magnesium alone.

A composition provided herein can be formulated to comprise an amount of a salt or complex containing magnesium such that a daily dose of magnesium between about 100 mg and about 1200 mg (e.g., between about 100 and about 1000 mg, between about 100 mg and about 800 mg, between about 100 mg and about 600 mg, between about 100 mg and about 500 mg, between about 100 mg and about 450 mg, between about 200 mg and about 1200 mg, between about 250 mg and about 1200 mg, between about 300 mg and about 1200 mg, between about 350 mg and about 1200 mg, between about 600 mg and about 1200 mg, between about 800 mg and about 1200 mg, between about 200 mg and about 600 mg, between about 250 mg and about 550 mg, between about 300 mg and about 500 mg, between about 350 mg and about 450 mg, and between about 375 mg and about 425 mg) is administered. In some embodiments, a composition provided herein can be formulated to contain an amount of a salt or complex containing magnesium such that a daily dose of magnesium between about 200 mg and about 600 mg (e.g., between about 250 mg and about 550 mg, between about 300 mg and about 500 mg, between about 350 mg and about 450 mg, and between about 375 mg and about 425 mg) is administered. For example, a composition provided herein can be formulated such that a daily dose of about 400 mg of magnesium is administered.

Feverfew:

Feverfew or the feverfew extract is provided as a pain relieving agent. The feverfew extract utilized in the migraine treatment formulation is derived using existing separation techniques used on T. parthenium. The feverfew extract is generated through an extraction process that utilizes the flowers and fruit bodies of T. parthenium. Feverfew or the feverfew extract contains parthenolide and tanetin which are the active constituents of the feverfew extract. Parthenolide is a sesquiterpene lactone of the germacranolide class of organic molecules that occur naturally in T. parthenium. Parthenolide functions primarily as an anti-inflammatory and an anti-hyperalergesic.

In certain embodiments, feverfew or feverfew extract is found ranging from about 5 milligrams to about 400 milligrams of the migraine treatment formulation. In other embodiments, comprises from about 20 milligrams to about 200 milligrams of feverfew or extract thereof.

In certain embodiments, parthenolide is found in a range comprising from about 0.0001% to about 10% wt/wt of the feverfew extract. In other embodiments, parthenolide is found in a range comprising from about 10 micrograms to about 1000 micrograms.

In other embodiments, parthenolide is found in a range comprising from about 50 micrograms to about 700 micrograms. In certain embodiments, 500 micrograms of parthenolide is present in the formulation.

In some embodiments, a composition provided herein comprises between about 1% and 50% by weight of feverfew or feverfew extract. For example, about 2% to about 50% by weight, about 4% to about 50% by weight, about 5% to about 50% by weight, about 6% to about 50% by weight, about 10% to about 50% by weight, about 20% to about 50% by weight, about 25% to about 50% by weight; about 1% to about 40% by weight, about 1% to about 39% by weight, about 1% to about 38% by weight, about 1% to about 37% by weight, about 1% to about 36% by weight, about 1% to about 35% by weight, about 1% to about 34% by weight, about 1% to about 33% by weight, about 1% to about 32% by weight, about 1% to about 31% by weight, about 1% to about 30% by weight, about 5% to about 30% by weight, about 6% to about 30% by weight, about 7.5% to about 30% by weight, and about 8% to about 30% by weight of a composition provided herein can be feverfew or feverfew extract. In some embodiments, a composition provided herein can be formulated to contain an amount of feverfew or feverfew extract of between about 5% to about 30% by weight (e.g., about 6% to about 29% by weight, about 7% to about 28% by weight, about 8% to about 26% by weight, about 9% to about 25% by weight, about 10% to about 24% by weight, and about 5% to about 30% by weight).

Typically, a composition provided herein comprises between about 20 mg to about 500 mg of feverfew or feverfew extract (e.g., about 25 mg to about 250 mg, about 25 mg to about 200 mg, about 25 mg to about 150 mg; about 50 mg to about 500 mg, about 80 mg to about 500 mg, about 85 mg to about 500 mg, about 90 mg to about 500 mg, about 95 mg to about 500 mg, about 100 mg to about 500 mg, about 125 mg to about 500 mg, about 150 mg to about 500 mg, about 200 mg to about 500 mg; about 50 mg to about 250 mg, about 75 mg to about 250 mg, about 80 mg to about 250 mg, about 90 mg to about 250 mg, about 95 mg to about 250 mg). In some embodiments, a composition provided herein can contain between about 50 mg to about 350 mg of feverfew or feverfew extract (e.g., about 75 mg to about 325 mg, about 80 mg to about 310 mg, about 90 mg to about 300 mg, about 95 mg to about 250 mg).

A composition provided herein can be formulated to comprise an amount of feverfew or feverfew extract such that a daily dose of between about 20 mg and about 1200 mg (e.g., between about 20 and about 1000 mg, between about 20 mg and about 800 mg, between about 20 mg and about 600 mg, between about 20 mg and about 500 mg, between about 20 mg and about 450 mg, between about 200 mg and about 1200 mg, between about 250 mg and about 1200 mg, between about 300 mg and about 1200 mg, between about 350 mg and about 1200 mg, between about 600 mg and about 1200 mg, between about 800 mg and about 1200 mg, between about 200 mg and about 600 mg, between about 250 mg and about 550 mg, between about 300 mg and about 500 mg, between about 350 mg and about 450 mg, and between about 375 mg and about 425 mg) of feverfew or feverfew extract is administered. In some embodiments, a composition provided herein can be formulated to contain an amount of feverfew or feverfew extract such that a daily dose of between about 20 mg and about 600 mg (e.g., between about 25 mg and about 550 mg, between about 30 mg and about 500 mg, between about 35 mg and about 450 mg, and between about 37.5 mg and about 425 mg) is administered.

5-Hydroxytryptophan:

5-HTP is a chemical by-product of the protein building block L-tryptophan. It is also produced commercially from the seeds of an African plant known as Griffonia simplicifoliais. 5-HTP is a precursor to the neurotransmitter serotonin. Serotonin plays a role in many important functions, including sleep, appetite, memory, learning, mood, sex, and endocrine regulation. 5-HTP has been suggested as a treatment for many conditions. (Natural Med. Journal, October 2011 Vol. 3 Issue 10). Some research supports the use of 5-HTP in treating cerebellar ataxia, headache, depression, psychiatric disorders, and fibromyalgia, and as an appetite suppressant or weight loss agent. There is insufficient scientific evidence to support the use of 5-HTP for any other medical condition.

In some embodiments, a composition provided herein comprises between about 1% and 50% by weight of 5-HTP. For example, about 2% to about 50% by weight, about 4% to about 50% by weight, about 5% to about 50% by weight, about 6% to about 50% by weight, about 10% to about 50% by weight, about 20% to about 50% by weight, about 25% to about 50% by weight; about 1% to about 40% by weight, about 1% to about 39% by weight, about 1% to about 38% by weight, about 1% to about 37% by weight, about 1% to about 36% by weight, about 1% to about 35% by weight, about 1% to about 34% by weight, about 1% to about 33% by weight, about 1% to about 32% by weight, about 1% to about 31% by weight, about 1% to about 30% by weight, about 5% to about 30% by weight, about 6% to about 30% by weight, about 7.5% to about 30% by weight, and about 8% to about 30% by weight of a composition provided herein can be 5-HTP. In some embodiments, a composition provided herein can be formulated to contain an amount of 5-HTP of between about 5% to about 30% by weight (e.g., about 6% to about 29% by weight, about 7% to about 28% by weight, about 8% to about 26% by weight, about 9% to about 25% by weight, about 10% to about 24% by weight, and about 5% to about 30% by weight).

Typically, a composition provided herein comprises between about 20 mg to about 500 mg of 5-HTP (e.g., about 25 mg to about 250 mg, about 25 mg to about 200 mg, about 25 mg to about 150 mg; about 50 mg to about 500 mg, about 80 mg to about 500 mg, about 85 mg to about 500 mg, about 90 mg to about 500 mg, about 95 mg to about 500 mg, about 100 mg to about 500 mg, about 125 mg to about 500 mg, about 150 mg to about 500 mg, about 200 mg to about 500 mg; about 50 mg to about 250 mg, about 75 mg to about 250 mg, about 80 mg to about 250 mg, about 90 mg to about 250 mg, about 95 mg to about 250 mg). In some embodiments, a composition provided herein can contain between about 50 mg to about 350 mg of 5-HTP (e.g., about 75 mg to about 325 mg, about 80 mg to about 310 mg, about 90 mg to about 300 mg, about 95 mg to about 250 mg).

A composition provided herein can be formulated to comprise an amount of 5-HTP such that a daily dose of between about 20 mg and about 1200 mg (e.g., between about 20 and about 1000 mg, between about 20 mg and about 800 mg, between about 20 mg and about 600 mg, between about 20 mg and about 500 mg, between about 20 mg and about 450 mg, between about 200 mg and about 1200 mg, between about 250 mg and about 1200 mg, between about 300 mg and about 1200 mg, between about 350 mg and about 1200 mg, between about 600 mg and about 1200 mg, between about 800 mg and about 1200 mg, between about 200 mg and about 600 mg, between about 250 mg and about 550 mg, between about 300 mg and about 500 mg, between about 350 mg and about 450 mg, and between about 375 mg and about 425 mg) of 5-HTP is administered. In some embodiments, a composition provided herein can be formulated to contain an amount of 5-HTP such that a daily dose of between about 20 mg and about 600 mg (e.g., between about 25 mg and about 550 mg, between about 30 mg and about 500 mg, between about 35 mg and about 450 mg, and between about 37.5 mg and about 425 mg) is administered.

### Methods of Treatment

The step of determining the onset of a migraine headache is provided as the determinant step that initiates the treatment method. The migraine treatment formulation is effective at reducing and stopping symptoms associated with a migraine during the initial migraine phases. Migraines occur in four phases the prodrome phase, the aura phase, the pain/headache phase, and the postdrome phase. The prodrome phases occurs hours or days before pain/headache phase and is characterized by a wide variety of phenomena that include but are not limited to, altered mood, irritability, depression or euphoria, fatigue, craving certain foods, stiff muscles, constipation or diarrhea, and sensitivity to smell or noises. The aura phase immediately precedes the pain/headache phase and is characterized by the visual distortion of sensitivity to light. The pain/headache phase is classic unilateral headache that characterizes a migraine. The postpodrome phase can last for several days following a migraine and is characterized by migraine symptoms that persist following the main migraine headache. By detecting the prodrome phase and/or the aura phase, the user can take the necessary steps to administer the migraine treatment formulation. The migraine treatment formulation is provided as a dosage form comprises one or more of a tablet, a caplet, gel capsule, powder capsule, gel, liquid, powder or transdermal patch.

In certain embodiments, a method of preventing or treating a migraine or other pain, comprises administering a therapeutic composition to a subject in need thereof, wherein the therapeutic composition comprises about 10% to about 70% weight/weight (w/w) of riboflavin, about 10% to about 60% w/w of magnesium, about 5% to about 30% w/w of feverfew, about 5% to about 30% w/w of 5-hydroxytryptophan (5-HTP) or combinations thereof.

In certain embodiments, a unit dose comprises: about 50-800 mg of riboflavin, about 50-600 mg magnesium oxide and/or magnesium salt of an organic salt, about 20-200 mg of feverfew or feverfew extract, about 20-200 mg of 5-hydroxytryptophan (5-HTP) or combinations thereof. In other embodiments, a unit dose comprises: about 200 mg of riboflavin, about 180 mg magnesium oxide and/or magnesium salt of an organic salt, about 50 mg of feverfew or feverfew extract, about 50 mg of 5-hydroxytryptophan (5-HTP) or combinations thereof. In certain embodiments, a unit dose consists of 200 mg of riboflavin, about 180 mg magnesium oxide and/or magnesium salt of an organic salt, about 50 mg of feverfew or feverfew extract and about 50 mg of 5-hydroxytryptophan (5-HTP).

For any agent used in the methods of the invention, the therapeutically effective amount or dose can be estimated initially from activity assays in cell cultures and/or animals. For example, a dose can be formulated in animal models to achieve a circulating concentration range that includes the IC₅₀ as determined by activity assays (e.g., the concentration of the test compound, which achieves a half-maximal inhibition of the proliferation activity). Such information can be used to more accurately determine useful doses in humans.

The dosage may vary depending upon the dosage form employed and the route of administration utilized. The exact formulation, route of administration and dosage can be chosen by the individual physician in view of the patient's condition. Dosage amount and interval may be adjusted individually to provide plasma levels of the active moiety which are sufficient to maintain therapeutic effects, termed the minimal effective concentration (MEC). Dosages necessary to achieve the MEC will depend on individual characteristics and route of administration. HPLC assays or bioassays can be used to determine plasma concentrations. Dosage intervals can also be determined using the MEC value. Preparations should be administered using a regimen, which maintains plasma levels above the MEC for 10-90% of the time, preferably between 30-90% and most preferably 50-90%. Depending on the severity and responsiveness of the condition to be treated, dosing can also be a single administration of a slow release composition described hereinabove, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, etc.

While a composition according to the invention is useful for treating or preventing a migraine or stress headache in a subject, the artisan will recognize that the composition can be used to treat additional conditions or disorders associated with pain. Pain is a heterogeneous disorder and includes all types of pain, including acute and persistent pain. In some embodiments, the composition is used to treat or prevent cluster headaches.

Additional conditions for which a composition of the invention can be used include, e.g., pain associated with jaw pain, facial pain, atypical facial pain, post-traumatic headache, cervical pain and muscle spasm, craniofacial neuralgia. neuropathic pain, diabetic neuropathy, fibromyalgia, pain associated with somatoform disorders, arthritic pain, cancer pain, neck pain, shoulder pain, back pain, cluster headaches, tension-type headache, migraine, herpes neuralgia, phantom limb pain, central pain, dental pain, NSAID-resistant pain, visceral pain, surgical pain, post-operative pain, bone injury pain, pain during labor and delivery, pain resulting from burns, post-partum pain, angina pain, genitourinary tract-related pain, neuropathic pain, diabetic neuropathy, fibromyalgia, pain associated with somatoform disorders, arthritic pain, cancer pain, neck pain, shoulder pain, back pain, cluster headaches, tension-type headache, migraine, herpes neuralgia, phantom limb pain, central pain, dental pain, NSAID-resistant pain, visceral pain, surgical pain, post-operative pain, bone injury pain, pain during labor and delivery, pain resulting from burns, including sunburn, post-partum pain, angina pain, and genitourinary tract-related pain including cystitis. In some embodiments, the composition is used to treat or prevent nociceptive pain.

The composition can be used to prevent a headache from beginning, to treat a migraine headache that has begun and/or to prevent or minimize a recurrence of a headache. Typically, the effectiveness of treatment is assessed by assessing a lessening of one or more traits associated with the condition. For example, for migraines one will evaluate one or more of maximal severity, associated vomiting, and migraine frequency. Effectiveness of treatment can also be monitored using additional methods known in the art, e.g., questionnaires used in clinical trials, which for example, assess: role-function restrictive (the degree to which performance of normal activities is restricted or limited by migraine); role function preventive (the degree to which performance of normal activities is presented or interrupted by migraine); and emotional function (the emotional effects of migraine). For example, typical questions address migraine-associated problems in attending social activities (role function restrictive) or the degree to which a migraine patient feels their migraines are a burden for others (emotional function). The MSQ has shown evidence of reliability and validity in migraine suffers participating in clinical trials.

### Pharmaceutical Compositions

The compositions of the invention may be consumed in any form that delivers an effective dose to a subject. In one embodiment, it is provided as a dietary supplement, e.g., a capsule, a tablet, a caplet, a liquid beverage, a powder beverage mix, a dietary gel, or as a ready-to-eat bar or drink product. The dosage form of a dietary supplement may be provided in accordance with customary processing techniques for herbal and/or dietary supplements, wherein the active ingredients are suitably processed and compressed into, e.g., one or more tablets and/or caplets, with suitable excipients.

The composition may further comprise natural and/or artificial flavoring components, dyes or other coloring additives, preservatives and other conventional food supplement additives known in the art. If desired, the pharmaceutical compositions may further contain additional components to further increase the speed and or ease with which the substances enter the bloodstream. A dietary supplement form of the composition may be provided in accordance with customary processing techniques for herbal and/or dietary supplements in any of the forms mentioned above. For instance, in the embodiment in which the supplemental composition is provided is a capsule, the active ingredients may be suitably processed and encapsulated into capsules, e.g., cellulose, gelatin, etc., or compressed into caplets with cellulose, croscarmellose sodium, stearic acid, magnesium stearate, silica and other with suitable excipients. Those of skill in the art will appreciate that the supplemental composition may contain a variety of excipients. Typically, a desired daily dosage is achieved by administering an appropriate quantity of unit dosages.

The preparation of the present invention may include excipient materials comprising: calcium carbonate, coloring agents, whiteners, preservatives, and flavors, triacetin, magnesium stearate, sterotes, natural or artificial flavors, essential oils, plant extracts, fruit essences, gelatins, or combinations thereof. Other examples of suitable excipients of this invention include, but are not limited to, anti-adherents, binders (e.g., macrocrystalline cellulose, gum tragacanth, or gelatin), coatings, disintegrants, fillers, diluents, softeners, emulsifiers, flavoring agents, coloring agents, adjuvants, lubricants, functional agents (e.g., nutrients), viscosity modifiers, bulking agents, glidiants (e.g., colloidal silicon dioxide) surface active agents, osmotic agents, diluents, or any other non-active ingredient, or combinations thereof.

Optionally the preparation of the present invention may include other artificial or natural sweeteners, bulk sweeteners, or combinations thereof. Bulk sweeteners include both caloric and non-caloric compounds. Non-limiting examples of bulk sweeteners include sucrose, dextrose, maltose, dextrin, dried invert sugar, fructose, high fructose corn syrup, levulose, galactose, corn syrup solids, tagatose, polyols (e.g., sorbitol, mannitol, xylitol, lactitol, erythritol, and maltitol), hydrogenated starch hydrolysates, isomalt, trehalose, and combinations thereof.

The preparation of pharmaceutical compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or other known methods.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

The compositions of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained. Furthermore, preferred compositions for the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, suppositories, ointments, lotions, aerosol sprays and gels, wherein the concentration of active ingredient would typically range from 0.01% to 60%, w/w or w/v.

The active compositions defined above, may be also formulated as suppositories, using for example, polyalkylene glycols, for example, propylene glycol, as the carrier. In some embodiments, suppositories are advantageously prepared from fatty emulsions or suspensions.

The compositions of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compositions of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, and triethanolamine oleate. The dosage regimen utilizing the molecules is selected in accordance with a variety of factors including type, species, age, weight, sex and medical condition of the patient; the severity of the condition to be treated; the route of administration; the renal and hepatic function of the patient; and the particular molecule or salt thereof employed. An ordinarily skilled physician or veterinarian can readily determine and prescribe the effective amount of the drug required to prevent, counter or arrest the progress of the condition. Compositions of the present invention may be administered in a single daily dose, or the total daily dosage may be administered in divided doses of two, three or four times daily.

The composition may alternatively be formulated for other modes of delivery, e.g., for sublingual, transnasal, parenteral, subtopical transepithelial, as a transdermal patch, subdermal, or inhalation delivery. Preparation for delivery in a transdermal patch can be performed using methods also known in the art, including those described generally in, e.g., U.S. Pat. Nos. 5,186,938; 6,183,770; 4,861,800; 4,284,444 and WO 89/09051. Patches can be made to control the release of skin-permeable active ingredients over a 12 hour, 24 hour, 3 day, and 7 day period. The penetration through skin of specific formulations may be measures by standard methods in the art (for example, Franz et al., J. Invest. Derm. 64:194-195 (1975)).

In one embodiment, the present invention includes a transdermal patch which comprises a pharmaceutically effective amount of a formulation embodied herein for delivery of the formulation to the bloodstream of a user. The patch may comprise a backing, and a skin-adhesive polymer matrix attached to one side of the backing. The patch may further optionally comprise a release liner removably attached to the skin-adhesive polymer matrix. The matrix may include a unit dose of the formulations embodied herein, a carrier agent, a terpene, and a permeation agent. Once attached to the skin, the formulation is capable of diffusing from the matrix into the bloodstream of the user in therapeutically effective amounts.

Transdermal drug delivery offers an advantageous mode of drug administration by eliminating first pass hepatic metabolism and providing sustained drug release for a prolonged period of time. It is painless when compared to needles and therefore offers superior patient compatibility. The skin permits the influx of a variety of small molecules that are fairly lipophilic (log P>1.5) and have molecular weight less than 500.

The composition may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage may be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

The composition can be provided as an article of manufacture in combination with labeling that the composition is to be taken for decreasing symptoms of a migraine headache or other pain as described herein. The article is typically a container or kit such as a bottle or box containing unit dosage forms (e.g., tablets, capsules) with labeling indicating the rate at which the unit dosage should be consumed.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

1. A therapeutic composition comprising: riboflavin, magnesium, feverfew, an amino acid, an amino acid precursor or combinations thereof.

2. The therapeutic composition of claim 1, wherein the amino acid is 5-hydroxytryptophan (5-HTP).

3. The therapeutic composition of claim 2, wherein the composition comprises: about 10% to about 70% weight/weight (w/w) of riboflavin, about 10% to about 60% w/w of magnesium, about 5% to about 30% w/w of feverfew, about 5% to about 30% w/w of 5-hydroxytryptophan (5-HTP) or combinations thereof.

4. The therapeutic composition of claim 2, wherein a unit dose comprises: about 100-500 mg of riboflavin, about 100-400 mg magnesium oxide and/or magnesium salt of an organic salt, about 20-200 mg of feverfew, about 20-200 mg of 5-hydroxytryptophan (5-HTP) or combinations thereof.

5. The therapeutic composition of claim 1, wherein the magnesium is provided as a magnesium oxide and/or a magnesium salt of an organic salt.

6. The therapeutic composition of claim 5, wherein the magnesium salt of an organic salt is magnesium citrate.

7. The therapeutic composition of claim 1, wherein a dosage form comprises one or more of a tablet, a caplet, gel capsule, powder capsule, gel, liquid, powder or transdermal patch.

8. The therapeutic composition of claim 1, further comprising one or more pharmaceutically acceptable carriers.

9. A therapeutic composition for treatment or prevention of migraines consisting essentially of: riboflavin, magnesium, feverfew and 5-hydroxytryptophan (5-HTP).

10. The therapeutic composition of claim 9, wherein the composition consists of about 10% to about 70% weight/weight (w/w) of riboflavin, about 10% to about 60% w/w of magnesium, about 5% to about 30% w/w of feverfew and about 5% to about 30% w/w of 5-hydroxytryptophan (5-HTP).

11. The therapeutic composition of claim 9, wherein a unit dose consists of about 100-500 mg of riboflavin, about 100-400 mg magnesium oxide and/or magnesium salt of an organic salt, about 20-200 mg of feverfew and about 20-200 mg of 5-hydroxytryptophan (5-HTP).

12. The therapeutic composition of claim 9, wherein the magnesium is provided as a magnesium oxide and/or a magnesium salt of an organic salt.

13. The therapeutic composition of claim 12, wherein the magnesium salt of an organic salt is magnesium citrate.

14. The therapeutic composition of claim 9, wherein a dosage form consists of one or more of a tablet, a caplet, gel capsule, powder capsule, gel, liquid, powder or transdermal patch.

15. A method of treating or preventing a migraine comprising administering a therapeutic composition to a subject in need thereof, the therapeutic composition comprising about 10% to about 70% weight/weight (w/w) of riboflavin, about 10% to about 60% w/w of magnesium, about 5% to about 30% w/w of feverfew, about 5% to about 30% w/w of 5-hydroxytryptophan (5-HTP) or combinations thereof.

16. The method of claim 15, wherein a unit dose comprises: about 100-500 mg of riboflavin, about 100-400 mg magnesium oxide and/or magnesium salt of an organic salt, about 20-200 mg of feverfew, about 20-200 mg of 5-hydroxytryptophan (5-HTP) or combinations thereof.

17. The method of claim 16, wherein the magnesium is provided as a magnesium oxide and/or a magnesium salt of an organic salt.

18. The method of claim 18, wherein the magnesium salt of an organic salt is magnesium citrate.

19. The method of claim 16, wherein a dosage form comprises one or more of a tablet, a caplet, gel capsule, powder capsule, gel, liquid, powder or transdermal patch.

20. A composition for the prevention and treatment of migraine headaches and associated symptoms thereof comprising:
an effective amount of riboflavin;
an effective amount of magnesium provided as magnesium oxide, magnesium salt of an organic salt, or combinations thereof;
an effective amount of feverfew or feverfew extract;
an effective amount of 5-hydroxytryptophan (5-HTP); and
an effective amount of at least one pharmaceutical acceptable carrier.

21. The therapeutic composition of claim 20, wherein the magnesium salt of an organic salt is magnesium citrate.
